(19) United States
(12) Patent Application Publication
(10) Pub. No.: US 2021/0238689 A1
(43) Pub. Date: Aug. 5, 2021

(54) TUMOR FUNCTIONAL MUTATION AND EPITOPE LOADS AS IMPROVED PREDICTIVE BIOMARKERS FOR IMMUNOTHERAPY RESPONSE

(71) Applicant: KONINKLIJKE PHILIPS N.V., EINDHOVEN (NL)

(72) Inventors: Yee Him Cheung, Boston, MA (US); Alexander Ryan Mankovich, Somerville, MA (US); Jie Wu, Cambridge, MA (US); Nevenka Dimitrova, Pelham Manor, NY (US)

(21) Appl. No.: 17/050,642

(22) PCT Filed: Apr. 16, 2019
(86) PCT No.: PCT/EP2019/059717

§ 371 Date: Oct. 26, 2020

Related U.S. Application Data
(60) Provisional application No. 62/662,357, filed on Apr. 25, 2018.

(51) Int. Cl. C12Q 1/6886; G16B 20/20; G16B 30/00

(52) U.S. Cl. CPC C12Q 1/6886; G16B 20/20; C12Q 2600/156; C12Q 2600/106; G16B 30/00

(57) ABSTRACT

A method (100, 200, 400) for predicting a response of a tumor to immunotherapy, comprising: analyzing (120) a tumor sample; analyzing (130) a non-tumor sample obtained from the patient; identifying (140) one or more tumor-specific mutations; analyzing (150) the genetic information from the tumor sample to determine a variant allele frequency for the identified tumor-specific mutations; analyzing (160) genetic information to determine a tumor purity of the patients tumor; determining (210) a pathogenicity for the identified tumor-specific mutations; calculating (220), from: (i) the determined variant allele frequency and/or a determined allele-specific expression, exon expression, or gene expression of the one or more tumor-specific mutations; (ii) the determined tumor purity; and (iii) the determined pathogenicity, a tumor functional mutation load score; predicting (410), based on the score, a response of the patients tumor to an immunotherapy treatment; and determining (420), based on said prediction, a treatment for the patient.

### FIELD OF THE DISCLOSURE

The present disclosure is directed generally to methods and systems for predicting the response of a tumor to immunotherapy.

### BACKGROUND

Immunotherapy can be an effective treatment for cancer, if the cancer cells are responsive to the specific immunotherapy treatment. If the cancer cells are not responsive to the specific immunotherapy treatment, the treatment can introduce patient toxicity and unwanted side-effects without providing any benefits. Accordingly, determining or estimating the responsiveness of a tumor to a specific immunotherapy treatment can be extremely beneficial when treating a patient.

Tumor mutation load and tumor neoantigen load are examples of predictive biomarkers for immunotherapy response. Tumor mutation load (TML), also called tumor mutation burden (TMB), can be defined as the total number of somatic, non-synonymous, exonic mutations in a tumor genome. This information can be derived, for example, by sequencing such as whole exome sequencing (WES), or can be estimated using targeted sequencing panels.

Tumor neoantigen load can be defined as the total number of predicted neoantigens in a sample. Some of the tumor-specific somatic mutations can result in mutated peptides or antigens that are presented on the major histocompatibility complex (MHC) molecules found at the surface of the tumor cells for potential recognition by the immune system. These tumor-specific antigens, called neoantigens, can be predicted, for example, by computational analysis.

In general, there is a linear positive correlation between the tumor mutation load and the tumor neoantigen load. Since a larger number of neoantigens represents a higher chance of inducing an anti-tumor immune response, the tumor neoantigen load is a useful biomarker for prediction of immunotherapy response, such as immune checkpoint blockade immunotherapy. Tumor mutation load is also a useful biomarker for prediction of immunotherapy response, and in some cases may serve as a proxy for the tumor neoantigen load.

However, the current methods and systems for predicting the response of a tumor to immunotherapy do not take into account the full complement of available information, and thus provide an incomplete prediction. For example, while tumor mutation load and tumor neoantigen load are known to be effective biomarkers for immunotherapy response, these methodologies treat all mutations as equals despite the fact that mutations will each have a different proportion within the tumor and will have varying functional impact.

### SUMMARY OF THE DISCLOSURE

There is a continued need for methods and systems that predict the response of a tumor to immunotherapy, while taking into account the specific mutations found within the tumor.

The present disclosure is directed to inventive methods and systems for predicting the response of a tumor to immunotherapy. Various embodiments and implementations herein are directed to two related methodologies that utilize information about tumor-specific mutations to generate a highly-accurate immunotherapy prediction. In both methodologies, genetic information about a tumor sample and about a non-tumor sample from a patient is obtained and analyzed. Tumor-specific mutations are identified by comparing the genomic information from the tumor sample to the genomic information from the non-tumor sample, and the frequencies of the tumor-specific mutations within the tumor are determined or estimated. The tumor sample is also analyzed to determine a tumor purity of the patient's tumor.

In the first methodology, a pathogenicity for the each tumor-specific mutations is determined or estimated. A tumor functional mutation load score is then calculated using a summation of variant-based measures that combine, with adjustment for the determined tumor purity, the determined variant allele frequency, the determined allelic/exon/gene expressions, and/or the determined pathogenicity for each of the tumor-specific mutations. The tumor functional mutation load score is utilized to predict a response of the patient's tumor to an immunotherapy treatment, and a course of treatment is selected or designed based on this prediction.

In the second methodology, a neoantigen score comprising a likelihood that the tumor-specific mutation will be presented as a neoantigen is calculated, a T-cell reactivity score comprising a likelihood that the mutation will be recognized by the patient's T cells is calculated, and/or a B-cell epitope score comprising a likelihood that the mutation will be recognized by the patient's B-cell receptors is calculated. A tumor neoepitope load score is then calculated using a summation of variant-based measures that combine, with adjustment for the determined tumor purity, the determined variant allele frequency, the determined allelic/exon/gene expression, the neoantigen score, the T-cell reactivity score, and/or the B-cell epitope score for each of the tumor-specific mutations. The tumor neoepitope load score is utilized to predict a response of the patient's tumor to an immunotherapy treatment, and a course of treatment is selected or designed based on this prediction.

Generally, in one aspect, a method for predicting a response of a tumor to immunotherapy is provided. The method includes: (i) analyzing a tumor sample obtained from a patient's tumor, comprising sequencing at least a portion of the genetic information of the tumor sample, wherein the tumor sample comprises a plurality of different genomes differentiated by one or more mutations, at least some of the mutations present at variable amounts within the tumor sample; (ii) analyzing a non-tumor sample obtained from the patient, comprising sequencing at least a portion of the genetic information of the non-tumor sample; (iii) identifying, by comparing the genetic information from the tumor sample to the genetic information from the non-tumor sample, one or more tumor-specific mutations found only in the tumor sample; (iv) analyzing the genetic information from the tumor sample to determine a variant allele frequency for the identified one or more tumor-specific mutations; (v) analyzing the genetic information from the tumor sample to determine a tumor purity of the patient's tumor; (vi) determining a pathogenicity for at least one of the identified one or more tumor-specific mutations; (vii) calculating, from: (1) the determined variant allele frequency and/or a determined allele-specific expression, exon expression, or gene expression of the one or more tumor-specific mutations; (2) the determined tumor purity; and (3) the determined pathogenicity, a tumor functional mutation load score for the at least one of the identified one or more tumor-specific mutations; (viii) predicting, based on the tumor functional mutation load score, a response of the patient's tumor to an immunotherapy treatment; and (ix) determining, based on said prediction, a treatment for the patient.

According to an embodiment, calculating the tumor functional mutation load score (Lₘ) comprises the equation:

$${L}_{m} = {\sum }_{i} [ f ( {v}_{i} , {a}_{i} , {e}_{i} ) \cdot {s}_{i} ]$$

where i is a tumor-specific mutation, ƒ is a function measuring a presence or expression of a variant based on measurements vᵢ, aᵢ and eᵢ, where vᵢ is the determined variant allele frequency (VAF), aᵢ is the allele-specific expression and eᵢ is the gene/exon expression of the tumor-specific mutation i. According to an embodiment, these measurements are adjusted for the purity of the specific sample, for example, by dividing their values by the determined tumor purity. sᵢ is the determined pathogenicity of the tumor-specific mutation i.

According to an embodiment, sᵢ=1 if no pathogenicity for mutation i is available.

According to an embodiment, the method further includes the step of obtaining a plurality of samples from the patient, including a sample from the patient's tumor and a non-tumor sample.

According to an embodiment, the step of determining a pathogenicity for a tumor-specific mutation comprises querying a pathogenicity database.

According to another aspect is a method for predicting a response of a tumor to immunotherapy. The method includes: (i) analyzing a tumor sample obtained from a patient's tumor, comprising sequencing at least a portion of the genetic information of the tumor sample, wherein the tumor sample comprises a plurality of different genomes differentiated by one or more mutations, at least some of the mutations present at variable amounts within the tumor sample; (ii) analyzing a non-tumor sample obtained from the patient, comprising sequencing at least a portion of the genetic information of the non-tumor sample; (iii) identifying, by comparing the genetic information from the tumor sample to the genetic information from the non-tumor sample, one or more tumor-specific mutations found only in the tumor sample; (iv) analyzing the genetic information from the tumor sample to determine a variant allele frequency for the identified one or more tumor-specific mutations; (v) analyzing the genetic information from the tumor sample to determine a tumor purity of the patient's tumor; (vi) determining one or more of: (1) a neoantigen score for the at least one of the identified one or more tumor-specific mutations, comprising a likelihood that the mutation will be presented as a neoantigen; (2) a T-cell reactivity score for the at least one of the identified one or more tumor-specific mutations, comprising a likelihood that the mutation will be recognized by the patient's T cells; and (3) a B-cell epitope score for the at least one of the identified one or more tumor-specific mutations, comprising a likelihood that the mutation will be recognized by the patient's B-cell receptors; (vii) calculating, from: (1) one or more of the neoantigen score, the T-cell reactivity score, and/or the B-cell epitope score; (2) the determined tumor purity; and (3) the determined variant allele frequency and/or a determined allele-specific expression, exon expression, or gene expression of the identified one or more tumor-specific mutations, a tumor neoepitope load score for the at least one of the identified one or more tumor-specific mutations; (viii) predicting, based on the tumor neoepitope load score, a response of the patient's tumor to an immunotherapy treatment; and (ix) determining, based on said prediction, a treatment for the patient.

According to an embodiment, calculating the tumor neoepitope load score (Lₙ) comprises the equation:

$${L}_{n} = {\sum }_{i} [ f ( {v}_{i} , {a}_{i} , {e}_{i} ) \cdot ( {n}_{i} \cdot {r}_{i} + {b}_{i} ) ]$$

where i is a tumor-specific mutation, ƒ is a function measuring a presence or expression of a variant based on measurements vᵢ, aᵢ and eᵢ, where vᵢ is the determined variant allele frequency (VAF), aᵢ is the allele-specific expression and eᵢ is the gene/exon expression of mutation i for the tumor-specific mutation i. According to an embodiment, these measurements are adjusted for the purity of the sample, for example, by dividing their values by the determined tumor purity. nᵢ is the neoantigen score, rᵢ is the T-cell reactivity score, and bᵢ is the B-cell epitope score.

According to an embodiment, the method further includes weighting a T-cell immune response for the tumor to produce a T-cell immune response weight, wherein the calculation of the tumor neoepitope load score further comprise the T-cell immune response weight. According to an embodiment, the method further includes weighting a B-cell immune response for the tumor to produce a B-cell immune response weight, wherein the calculation of the tumor neoepitope load score further comprise the B-cell immune response weight. According to an embodiment, calculating the tumor neoepitope load score (Lₙ) comprises the equation:

$${L}_{n} = {\sum }_{i} [ f ( {v}_{i} , {a}_{i} , {e}_{i} ) \cdot ( {w}_{t} \cdot {n}_{i} \cdot {r}_{i} + {w}_{b} \cdot {b}_{i} ) ]$$

where i is a tumor-specific mutation, ƒ is a function measuring a presence or expression of a variant based on measurements vᵢ, aᵢ and eᵢ, where vᵢ is the determined variant allele frequency (VAF), aᵢ is the allele-specific expression and eᵢ is the gene/exon expression of mutation i for the tumor-specific mutation i. According to an embodiment, these measurements are adjusted for the purity of the sample, for example, by dividing their values by the determined tumor purity. nᵢ is the neoantigen score, rᵢ is the T-cell reactivity score, bᵢ is the B-cell epitope score, wₜ is the T-cell immune response weight, and w_{b} is the B-cell immune response weight.

According to an embodiment, the method further includes analyzing the tumor sample or non-tumor sample to characterize the patient's HLA type, where the neoantigen score is based at least in part on the patient's HLA type.

According to an aspect is a system configured to predict a response of a tumor to immunotherapy. The system includes: a processor configured to: (i) identify, by comparing genetic information from a tumor sample to genetic information from a non-tumor sample, one or more tumor-specific mutations found only in the tumor sample; (ii) analyze the genetic information from the tumor sample to determine a variant allele frequency for the identified one or more tumor-specific mutations; (iii) analyze the genetic information from the tumor sample to determine a tumor purity of the patient's tumor; (iv) determine a pathogenicity for at least one of the identified one or more tumor-specific mutations; (v) calculate, from: (1) the determined variant allele frequency and/or a determined allele-specific expression, exon expression, or gene expression of the one or more tumor-specific mutations; (2) the determined tumor purity; and (3) the determined pathogenicity, a tumor functional mutation load score for the at least one of the identified one or more tumor-specific mutations; and (vi) predict, based on the tumor functional mutation load score, a response of the patient's tumor to an immunotherapy treatment; and a user interface configured to provide said prediction to a user.

According to an embodiment, the system includes a pathogenicity database, where the processor is configured to determine a pathogenicity using data from the pathogenicity database.

According to an aspect is a system configured to predict a response of a tumor to immunotherapy. The system includes: a processor configured to: (i) identify, by comparing genetic information from a tumor sample to genetic information from a non-tumor sample, one or more tumor-specific mutations found only in the tumor sample; (ii) analyze the genetic information from the tumor sample to determine a variant allele frequency for the identified one or more tumor-specific mutations; (iii) analyze the genetic information from the tumor sample to determine a tumor purity of the patient's tumor; (iv) determine one or more of a neoantigen score for the at least one of the identified one or more tumor-specific mutations, comprising a likelihood that the mutation will be presented as a neoantigen, a T-cell reactivity score for the at least one of the identified one or more tumor-specific mutations, comprising a likelihood that the mutation will be recognized by the patient's T cells, and a B-cell epitope score for the at least one of the identified one or more tumor-specific mutations, comprising a likelihood that the mutation will be recognized by the patient's B-cell receptors; (v) calculate, from: (1) one or more of the neoantigen score, the T-cell reactivity score, and/or the B-cell epitope score; (2) the determined tumor purity; and (3) the determined variant allele frequency and/or a determined allele-specific expression, exon expression, or gene expression of the identified one or more tumor-specific mutations, a tumor neoepitope load score for the at least one of the identified one or more tumor-specific mutations; and (vi) predict, based on the tumor neoepitope load score, a response of the patient's tumor to an immunotherapy treatment; and a user interface configured to provide said prediction to a user.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for predicting the response of a tumor to immunotherapy, in accordance with an embodiment.

FIG. 2 is a flowchart of a method for predicting the response of a tumor to immunotherapy, in accordance with an embodiment.

FIG. 3 is a flowchart of a method for predicting the response of a tumor to immunotherapy, in accordance with an embodiment.

FIG. 4 is a flowchart of a method for predicting the response of a tumor to immunotherapy, in accordance with an embodiment.

FIG. 5 is a flowchart of a method for predicting the response of a tumor to immunotherapy, in accordance with an embodiment.

FIG. 6 is a flowchart of a method for determining tumor purity, in accordance with an embodiment.

FIG. 7 is a schematic representation of a system for predicting the response of a tumor to immunotherapy, in accordance with an embodiment.

FIG. 8 is a schematic representation of tumor purity, in accordance with an embodiment.

### DETAILED DESCRIPTION OF EMBODIMENTS

The present disclosure describes various embodiments of a system and method for incorporating information about tumor-specific mutations into immunotherapy decision. More generally, Applicant has recognized and appreciated that it would be beneficial to provide a system that predicts the response of a tumor to immunotherapy. Using the system, genetic information about a tumor sample and about a non-tumor sample from a patient is obtained and analyzed. Tumor-specific mutations are identified by comparing the genomic information from the tumor sample to the genomic information from the non-tumor sample, and the frequencies of the tumor-specific mutations within the tumor are determined or estimated. The tumor sample is also analyzed to determine a tumor purity of the patient's tumor.

According to a first embodiment, a pathogenicity for the each tumor-specific mutations is determined or estimated. A tumor functional mutation load score is then calculated using a summation of the determined frequency, the determined tumor purity, and the determined pathogenicity for each of the tumor-specific mutations. The tumor functional mutation load score is utilized to predict a response of the patient's tumor to an immunotherapy treatment, and a course of treatment is selected or designed based on this prediction.

According to a second embodiment, a neoantigen score comprising a likelihood that the tumor-specific mutation will be presented as a neoantigen is calculated, a T-cell reactivity score comprising a likelihood that the mutation will be recognized by the patient's T cells is calculated, and a B-cell epitope score comprising a likelihood that the mutation will be recognized by the patient's B-cell receptors is calculated. A tumor neoepitope load score is then calculated using a summation of variant-based measures that combine, with adjustment for the determined tumor purity, the determined variant allele frequency and/or the determined allelic/exon/gene expressions, the neoantigen score, the T-cell reactivity score, and/or the B-cell epitope score for each of the tumor-specific mutations. The tumor neoepitope load score is utilized to predict a response of the patient's tumor to an immunotherapy treatment, and a course of treatment is selected or designed based on this prediction.

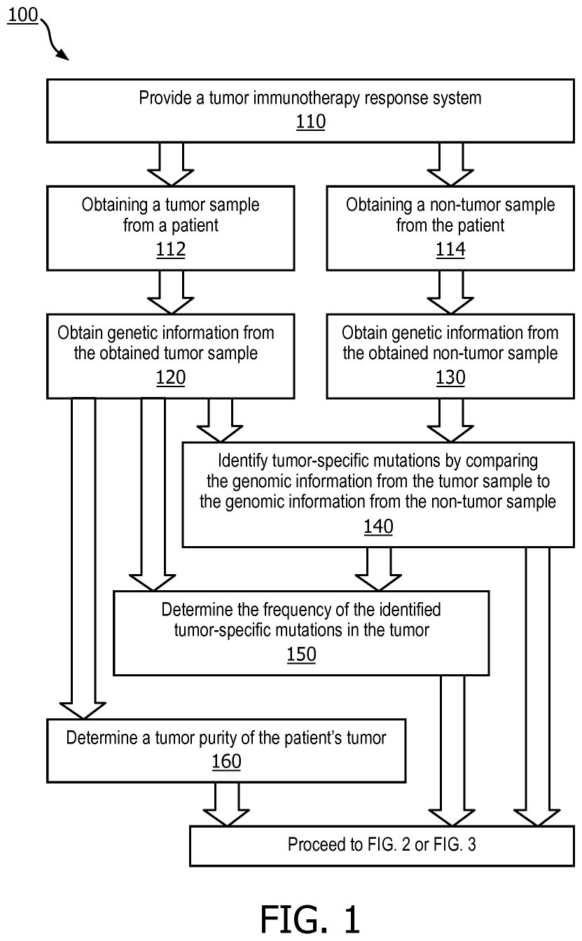

Referring to FIG. 1, in one embodiment, is a flowchart of a method 100 for predicting the response of a tumor to immunotherapy. The methods described in connection with the figures are provided as examples only, and shall be understood not to limit the scope of the disclosure. At step 110 of the method, a system configured or designed to provide a tumor immunotherapy response prediction or estimate is provided. The tumor immunotherapy response prediction or estimate system can be any of the systems described or otherwise envisioned herein.

At step 112 of the method, a tumor sample is obtained from a patient. The tumor sample may be any sample obtained from a patient's tumor, or from a tissue or location suspected to be or comprise a tumor. Tumor can be defined, for example, as a plurality of cancerous cells, and can be concentrated or diffuse. The tumor sample may be collected using any method or system for cell collection, such as through a biopsy or other tumor collection method.

At step 114 of the method, a non-tumor sample is obtained from the patient. The non-tumor sample may be provided from any location or tissue from the patient, preferably any location or tissue that is not likely to contain tumor cells. For example, the non-tumor sample may be skin cells, blood cells, saliva cells, or any other type of cells. The non-tumor sample may be collected using any method or system for cell collection.

At step 120 of the method, the tumor sample obtained from the patient is analyzed by sequencing at least a portion of the genomic information of the tumor sample. Genetic material such as DNA and RNA is extracted from the cancer cells obtained from the tumor, and the genetic material is sequenced. The sequencing can be whole genome sequencing, whole exome sequencing, targeted exome sequencing, targeted SNP analysis, and/or any other type of sequencing. The sequencing may be designed to enable variant allele frequency detection and/or quantification based on, for example, a fraction of reads that carry a mutant allele. In this way, the sequencing identifies mutations found within the tumor sample and can simultaneously quantify the prevalence of those mutations within the tumor sample.

According to an embodiment, the tumor sample comprises a plurality of different genomes differentiated by one or more mutations, where at least some of the mutations are present at variable amounts within the tumor sample. It is well-known in the art that the development of cancer is facilitated by genetic mutations. Additionally, it is well-known in the art that that more mutations arise in the cancerous cells as the disease progresses. The rapid, unchecked multiplication of cells results in mutations that can enhance the progression of the disease. These mutations also serve as markers or identifiers of the cancer, and can serve as a target for cancer treatment.

The genetic information obtained by sequencing can be utilized immediately and/or can be stored for downstream analysis. The genetic information can be obtained by a sequencer as part of the tumor immunotherapy response prediction or estimate system, or can be obtained by a separate sequencer and communicated to the tumor immunotherapy response prediction or estimate system.

At step 130 of the method, the non-tumor sample obtained from the patient is analyzed by sequencing at least a portion of the genomic information of the non-tumor sample. Genetic material such as DNA and RNA is extracted from the non-cancerous cells obtained from the patient, and the genetic material is sequenced. The sequencing can be whole genome sequencing, whole exome sequencing, targeted exome sequencing, targeted SNP analysis, and/or any other type of sequencing. The genetic information obtained by sequencing can be utilized immediately and/or can be stored for downstream analysis. According to an embodiment, the non-tumor sample is sequenced using the same platform or sequencing methodology as used for the tumor sample to allow for more comprehensive comparison of the tumor and non-tumor samples. The genetic information can be obtained by a sequencer as part of the tumor immunotherapy response prediction or estimate system, or can be obtained by a separate sequencer and communicated to the tumor immunotherapy response prediction or estimate system.

At step 140 of the method, the genetic information obtained from the tumor sample is compared to the genetic information from the non-tumor sample. This can be performed using any method for comparing genetic information. The genetic information from the two samples can be compared directly, and/or can be compared to a reference sequence. This comparison will identify one or more mutations that are found only within the tumor sample. These mutations may be exonic mutations, or may be non-exonic mutations.

At step 150 of the method, the genetic information obtained from the tumor sample is analyzed to determine a frequency of the identified mutations found only within the tumor sample. For example, the variant allele frequency (VAF) of the identified mutations can be obtained from the sequencing information obtained from the tumor sample. This information may be obtained during sequencing of the genetic material from the tumor sample, or may be obtained after sequencing by analyzing stored sequencing information. According to one embodiment, the allele frequency is determined or estimated by quantifying, tracking, or otherwise counting the percentage of reads that encompass the location of a mutation and that comprise the mutant allele, relative to the percentage of reads that encompass the location of a mutation and do not comprise the mutant allele. Many other methods for determining, estimating, or otherwise quantifying allele frequencies are possible.

At step 160 of the method, the genetic information obtained from the tumor sample is analyzed to determine or characterize a tumor purity of the patient's tumor. Tumor purity can be defined, for example, as the intra-tumor heterogeneity or mixture of cancerous versus non-cancerous cells, and/or the intra-tumor heterogeneity or mixture of subpopulations of cancerous cells. These subpopulations may be characterized, for example, by different mutations. Tumor purity can be estimated, calculated, or otherwise characterized by analysis of the genomic data by a pathologist and/or by one or more algorithms. For example, an algorithm can be programmed, trained, or designed to calculate the most likely collection of genomes and their proportions in a sample using mutations, copy number aberrations, and/or other markers to distinguish between subpopulations. Consideration of tumor purity can be an important component of immunotherapy. If a tumor sample comprises numerous subpopulations, consideration of only one or some of the populations may yield misleading information about the outcomes of immunotherapy.

Method 100 therefore results in genetic information from tumor and non-tumor samples from a patient, and provides: (1) a characterization of tumor purity; (2) identification of one or more tumor-specific mutations; and (3) frequency information for the identified tumor-specific mutations. This information is utilized in methods 200 and 300, described below.

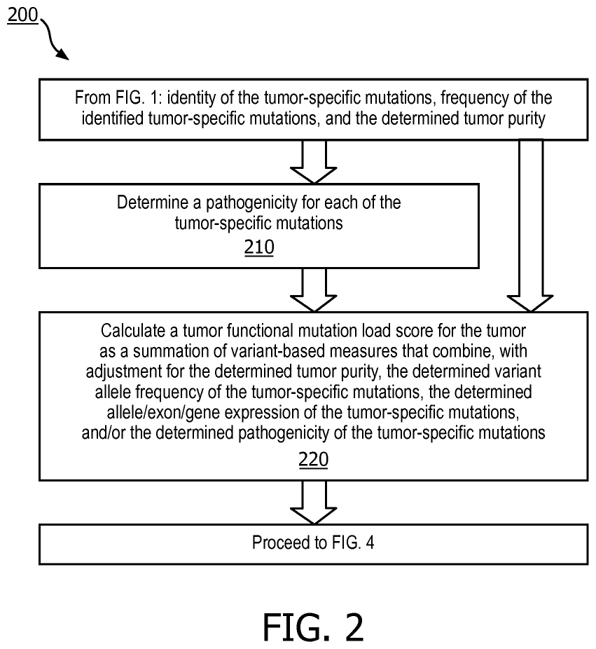

Referring to FIG. 2, in one embodiment, is a flowchart of a method 200 for predicting the response of a tumor to immunotherapy. The method begins with input information such as the information obtained via method 100, including a characterization of tumor purity, identification of one or more tumor-specific mutations, and frequency information for the identified tumor-specific mutations. Method 200 can utilize the tumor immunotherapy response prediction or estimate system described or otherwise envisioned herein, among other possible systems.

At step 210 of the method, the system determines a pathogenicity for the identified one or more tumor-specific mutations. Pathogenicity may be defined, for example, as a measurement or characterization of a mutation's effect on maintenance of cancer, progression of cancer, or resistance of a cancer to treatment, among other possible definitions. Pathogenicity may be based on any available information about a mutation. Pathogenicity may also or alternatively be based on analysis of the mutation and comparison to similar mutations. For example, a mutation may not have pathogenicity information available, or may not have sufficient pathogenicity information available, but a modeler, classifier, or algorithm may determine that the mutation is sufficiently similar to another mutation such that the pathogenicity will also be similar. Thus, the pathogenicity may be based on a classification of the mutation.

According to an embodiment, the system may query or otherwise communicate with a database of information about mutations associated with pathogenicity information. For example, the system may connect with or otherwise query or obtain information from a remote database. According to another embodiment, the system may comprise such a database. The database may comprise a list of mutations and information about the pathogenicity of each of these mutations. Notably, the database may indicate that there is no known pathogenicity associated with a particular mutation. Many other methods of retrieving, deriving, or generating information about the pathogenicity of mutations are possible.

For example, the pathogenicity may be determined using known pathogenicity analysis methodologies such as SIFT, PolyPhen2, GERP, PhyloP, and others. The pathogenicity score of one or more of these methodologies may be weighted and/or combined to produce a single score. The pathogenicity score may also be normalized.

At step 220 of the method, the system calculates a tumor functional mutation load score as a summation of information about: (i) the determined tumor purity; (ii) the determined variant allele frequency information for the identified tumor-specific mutations and/or allele, exon, and/or gene expression for the identified tumor-specific mutations; and/or (iii) the determined pathogenicity of the tumor-specific mutations. For example, according to one embodiment, a tumor functional mutation load score (Lₘ) is calculated using the following equation:

Lₘ=Σᵢ[ƒ(vᵢ,aᵢ,eᵢ)·sᵢ] (Eq. 1)

where: i is the index for a tumor-specific mutation identified in the tumor sample; ƒ is a function that measures the presence or expression of a variant based on any combinations of the measurements vᵢ, aᵢ and eᵢ depending on their availability and the user's choice; vᵢ is the variant allele frequency (VAF), aᵢ is the allele-specific expression and eᵢ is the gene/exon expression of mutation i. According to an embodiment, the following are some examples of function ƒ. For example, ƒ=vᵢ, if expression data is not available. As another example, ƒ=aᵢ, if allele-specific expression is available. As yet another example, ƒ=vᵢ·eᵢ, if allele-specific expression is not available and assuming that the expression of the allele is proportional to the fraction of cells that carry the alternative allele and the overall expression of the gene/exon.

According to an embodiment, all these measures should be adjusted for the tumor purity of the sample; and sᵢ is a normalized pathogenicity/conservation score. A higher score should indicate stronger functional impact or damaging effect of a mutation. The value of sᵢ can be set to one if it is not available. As shown in Eq. 1, the tumor functional mutation load score (Lₘ) is a summation of the relevant information about one or more tumor-specific mutations identified in the tumor sample.

Accordingly, the tumor functional mutation load score measures the effect of individual mutations by the product of their fractional presence and predicted functional impact. The aggregate effect of all mutations is then given by the sum of their products.

According to an embodiment, method 200 therefore results in a tumor functional mutation load score (Lₘ) that can be utilized to predict or estimate the response of the patient's sampled tumor to immunotherapy, as described in reference to method 400 below.

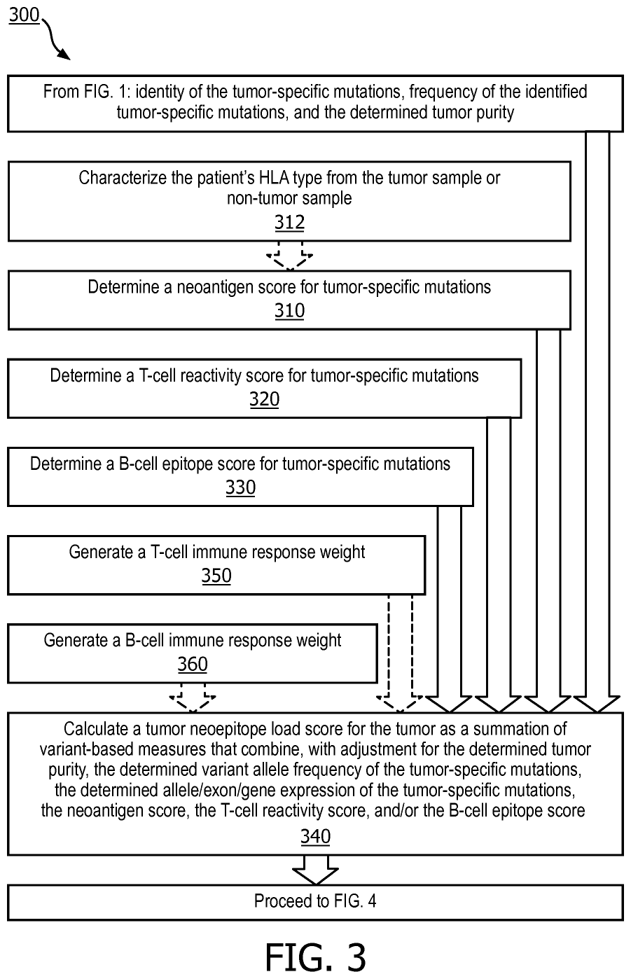

Referring to FIG. 3, in one embodiment, is a flowchart of a method 300 for predicting the response of a tumor to immunotherapy. The method begins with input information such as the information obtained via method 100, including a characterization of tumor purity, identification of one or more tumor-specific mutations, and frequency information for the identified tumor-specific mutations. Method 300 can utilize the tumor immunotherapy response prediction or estimate system described or otherwise envisioned herein, among other possible systems.

At step 310 of the method, the system determines a neoantigen score for the identified tumor-specific mutations, where the neo antigen score comprises a likelihood that the mutation will be presented at the surface of tumor cells as a neoantigen. According to an embodiment, the neoantigen score is a binary value with a value of one indicating a predicted neoantigen mutation (meaning that the mutation will be presented at the surface of tumor cells), and a value of zero indicating that the mutation is not a neoantigen mutation (meaning that the mutation will not be presented at the surface of tumor cells). According to an embodiment, the neoantigen score can be estimated computationally using the patient's HLA type and/or bioinformatics tools such as EpiJen, WAPP, NetCTL, and/or NetCTLpan, among other tools or algorithms. According to an embodiment, the neo antigen score can be set to one or otherwise ignored if the information is not available or otherwise cannot be determined.

According to an optional embodiment, at step 312 of the method the system characterizes the patient's HLA type from the tumor sample and/or the non-tumor sample. The patient's HLA type can be automatically determined from NGS data using tools such as OptiType, Polysolver, PHLAT, and/or HLAforest, among other tools or algorithms. This information can then be utilized in step 310 of the method when calculating a neoantigen score for a tumor-specific mutation.

At step 320 of the method, the system determines a T-cell reactivity score for the identified tumor-specific mutations, where the T-cell reactivity score comprises a likelihood that the mutation will be recognized by the patient's T cells to induce an anti-tumor immune response. According to an embodiment, the T-cell reactivity score is a binary value with a value of one indicating that the mutation is predicted to produce an immune response and a value of zero indicating that the mutation is predicted to not result in an immune response. Among many other methods, the T-cell reactivity score can be calculated or inferred using bioinformatics tools or algorithms such as POPI and/or POPISK, among others. According to an embodiment, the T-cell reactivity score can be set to one or otherwise ignored if the information is not available or otherwise cannot be determined.

At step 330 of the method, the system determines a B-cell epitope score for the identified tumor-specific mutations, where the B-cell epitope score comprises a likelihood that the mutation will be recognized by the patient's B-cell receptors. The B-cell receptor is a membrane-bound immunoglobulin with a wide range of antigen specificities, and each B-cell produces immunoglobulin of a single specificity. According to an embodiment, the B-cell epitope score is a binary value with a value of one indicating that the mutation is predicted to be recognized by the patient's B-cell receptors, and a value of zero indicating that the mutation is predicted to not be recognized by the patient's B-cell receptors. Among many other methods, the B-cell epitope score can be calculated or inferred using bioinformatics tools or algorithms such as COBEpro, BCPRed, and/or FBCPred for continuous-sequence epitopes (˜85% of documented B-cell epitopes), and EPMeta for discontinuous-sequence epitopes, among others. According to an embodiment, the B-cell epitope score can be set to one or otherwise ignored if the information is not available or otherwise cannot be determined.

At step 340 of the method, the system calculates a tumor neoepitope load score as a summation of the information about the determined tumor purity, the frequency and expression information for the identified tumor-specific mutations, the calculated neoantigen score, the T-cell reactivity score, and/or the B-cell epitope score, as described or otherwise envisioned herein.

According to an embodiment, a tumor neoepitope load score (Lₙ) is calculated using the following equation:

Lₙ=Σᵢ[ƒ(vᵢ,aᵢ,eᵢ)·(nᵢ·rᵢ+bᵢ)] (Eq. 2)

where i is the index for a tumor-specific mutation identified in the tumor sample; ƒ is a function that measures the presence or expression of a variant based on any combinations of the measurements vᵢ, aᵢ and eᵢ depending on their availability and the user's choice, where vᵢ is the variant allele frequency (VAF), aᵢ is the allele-specific expression and eᵢ is the gene/exon expression of mutation i; nᵢ is the neoantigen score; rᵢ is the T-cell reactivity score; and bᵢ is the B-cell epitope score. All these measures can be adjusted for tumor purity of the sample. According to an embodiment, the following are some examples of function ƒ. For example, ƒ=vᵢ, if expression data is not available. As another example, ƒ=aᵢ, if allele-specific expression is available. As yet another example, ƒ=vᵢ·eᵢ, if allele-specific expression is not available and assuming that the expression of the allele is proportional to the fraction of cells that carry the alternative allele and the overall expression of the gene/exon.

Accordingly, the tumor neoepitope load score measures the ability of inducing an immune response of an individual mutation by the product of its fractional presence and an epitope prediction score, which can be a weighted average of the T-cell (nᵢ·rᵢ) and B-cell (bᵢ) immunogenicities. For T-cell immune response, it mainly consists of the integrated antigen processing by HLA pathways and T-cell recognition and reaction. Unlike T-cells, B-cells can recognize soluble antigen for which their B-cell receptor is specific, and then process the antigen and present peptides using MHC class II molecules. The aggregate effect of all mutations is then given by the sum of their products. According to an embodiment, the formula for the epitope prediction score can be replaced by any other formula that can effectively measure the immune response predicted to be induced by a mutation.

According to an embodiment, the tumor neoepitope load score may further comprise user-defined weightings for respectively the T-cell and B-cell immune response. The value of these weightings can depend on factors such as the relative importance of T-cell and B-cell in a specific disease, the assumption and hypothesis of the analysis, the robustness of the prediction scores, and other factors. For example, if the assumption is that the immune response in the study is only contingent upon T-cell reactivity and the involvement of B-cell is negligible, then the user can set the T-cell weighting to 1 and the B-Cell weighting to 0.

Accordingly, at optional step 350 of the method, the system determines a weighting factor for the T-cell immune response for the tumor, thereby producing a T-cell immune response weight. This can be defined by the user based on, for example, the identity of the tumor-specific mutation, among other methods.

Similarly, at optional step 360 of the method, the system determines a weighting factor for the B-cell immune response for the tumor, thereby producing a B-cell immune response weight. This can be defined by the user based on, for example, the identity of the tumor-specific mutation, among other methods.

According to an embodiment calculation of the tumor neoepitope load score at step 340 of the method further comprises the T-cell immune response weight and the B-cell immune response weight. Accordingly, the tumor neoepitope load score (Lₙ) may be calculated using the following equation:

Lₙ=Σᵢ[ƒ(vᵢ,aᵢ,eᵢ)·(wₜ·nᵢ·rᵢ+w_{b}·bᵢ)] (Eq. 3)

where wₜ is the T-cell immune response weight and w_{b} is the B-cell immune response weight. If the immune response in the study is only contingent upon T-cell reactivity and the involvement of B-cells is negligible, then the user can set wₜ=1 and w_{b}=0. Similarly, if the immune response in the study is only contingent upon B-cell involvement and the involvement of T-cells is negligible, then the user can set wₜ=0 and w_{b}=1. Thus, wₜ and w_{b} can be set to any value between and including 0 and 1.

According to an embodiment, method 300 therefore results in a tumor neoepitope load score (Lₙ) that can be utilized to predict or estimate the response of the patient's sampled tumor to immunotherapy, as described in reference to method 400 below.

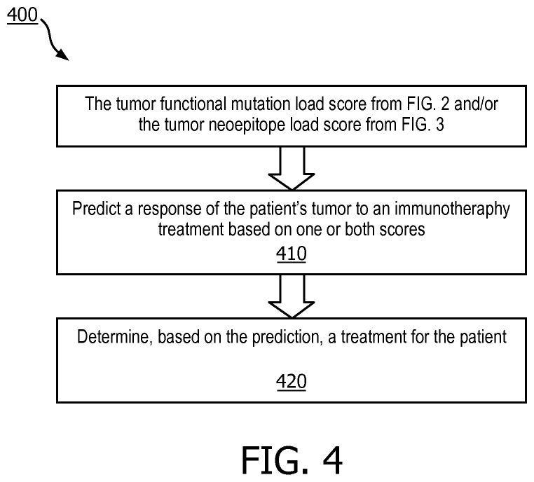

Referring to FIG. 4, in one embodiment, is a flowchart of a method 400 for predicting the response of a tumor to immunotherapy. The method begins with input information such as the tumor functional mutation load score (Lₘ) calculated in method 200, and/or the tumor neoepitope load score (Lₙ) calculated in method 300. Method 400 can utilize the tumor immunotherapy response prediction or estimate system described or otherwise envisioned herein, among other possible systems.

At step 410 of the method, the system predicts, based on the tumor functional mutation load score and/or the tumor neoepitope load score, the response of the patient's tumor to an immunotherapy treatment. According to an embodiment, the output of the tumor functional mutation load score and/or the tumor neoepitope load score is a number or other value that translates directly into a predicted response of the patient's tumor to an immunotherapy treatment. According to another embodiment, the output of the tumor functional mutation load score and/or the tumor neoepitope load score is a number or other value that undergoes additional analysis or interpretation to provide a predicted response of the patient's tumor to an immunotherapy treatment.

At step 420 of the method, a physician, clinician, or other user utilizes the prediction from step 410 to generate or otherwise inform a treatment for the patient. For example, the prediction may indicate that treatment X would be unlikely to generate a sufficient immune response in the tumor. Similarly, the prediction may indicate that treatment Y would be likely to generate a sufficient immune response in the tumor. Thus, the physician or clinician may use the prediction to select treatment Y over treatment X.

According to one example implementation of the methods described or otherwise envisioned herein, a clinician plans to use anti-PD1 immunotherapy for a cancer patient, but wants to predict the therapeutic response first using the tumor neoepitope load score. A tissue biopsy from the tumor bulk and a blood sample are taken from the patient, and whole exome sequencing (WES) is performed on both the tumor sample and blood sample. By running read alignment and variant calling on the generated sequencing data, somatic mutations can be identified along with their variant allele frequencies (VAF) using the blood sample as the matched normal reference. Tumor purity of the biopsy is computed using the WES data. The immunogenicity of each somatic mutation is further evaluated computationally to obtain the values of nᵢ and rᵢ using a combination of immunoinformatics tools. Since the effectiveness of anti-PD1 therapy is mainly dependent on the immune response of T-cells, wₜ is set to and w_{b} is set to 0. The patient's tumor neoepitope load score is calculated using Equation 2 or 3. The resulting tumor neoepitope load score is higher than 75% of the patient's cohort with the same diagnosis, clinical stage, and age range, indicating a good chance of positive response to anti-PD1 therapy in the patient. The clinician then decides to implement anti-PD1 immunotherapy for the patient.

According to another example implementation of the methods described or otherwise envisioned herein, the effectiveness of therapy may require a coordinated immune response from both T-cells and B-cells. In the case of allogeneic hematopoietic stem cell transplantation (alloSCT), a patient is administered chemotherapy and radiotherapy, after which the patient receives an infusion of hematopoietic stem cells of a compatible donor. A benefit of this infusion is the graf-versus-leukemia (GvL) effect, whereby the donor cells exhibit an immune response to residual malignant cells. Studies have demonstrated that this effect can be partially explained by, in one such case, a coordinated CD4+T- and B-cell response against autosomal antigen PTK2B. In this case, wₜ can be set to 0.5 and w_{b} can be set to 0.5, or another such weighting split between the two immune responses to better estimate the tumor neoepitope load score.

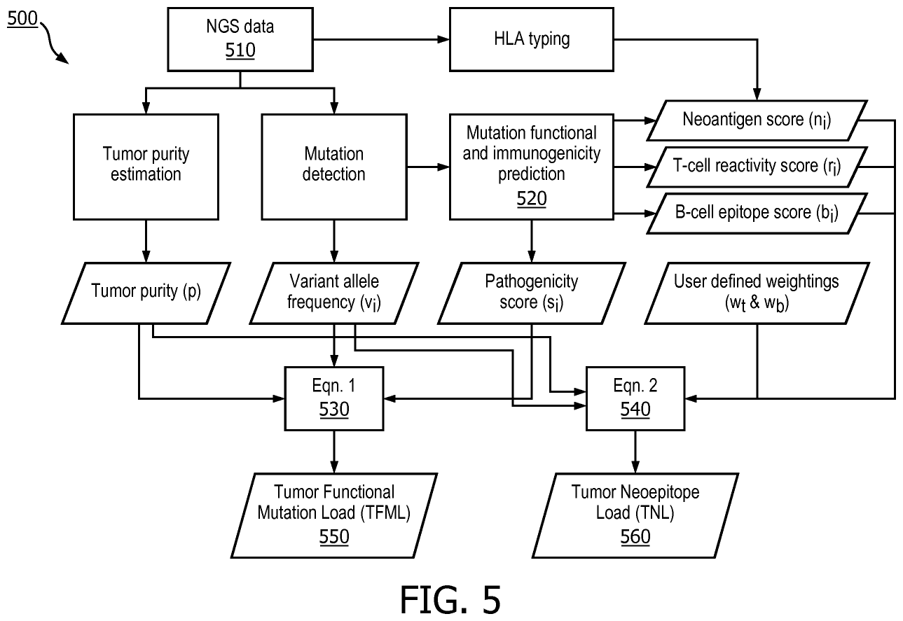

Referring to FIG. 5, in one embodiment, is a flowchart 500 for calculation of a tumor functional mutation load score (TFML) 550 and a tumor neoepitope load score (TNL) 560. According to an embodiment, a tumor immunotherapy response prediction or estimate system utilizes the workflow, input, and/or components of flowchart 500 to generate a tumor functional mutation load score or a tumor neoepitope load score. According to an embodiment, the system receives as input a tumor sample and a non-tumor sample, and/or the genetic information 510 obtained from the tumor sample and a non-tumor sample. The system may also receive pathogenicity information 520 as input, or may comprise a database containing pathogenicity information. The system utilizes the depicted input and Equation 1 (530) to generate a tumor functional mutation load score 550. The system utilizes the depicted input and Equation 2 or 3 (540) to generate a tumor neoepitope load score 560. Notably, not every component/step/element contained within flowchart 500 is required for either the tumor functional mutation load score (TFML) or the tumor neoepitope load score (TNL).

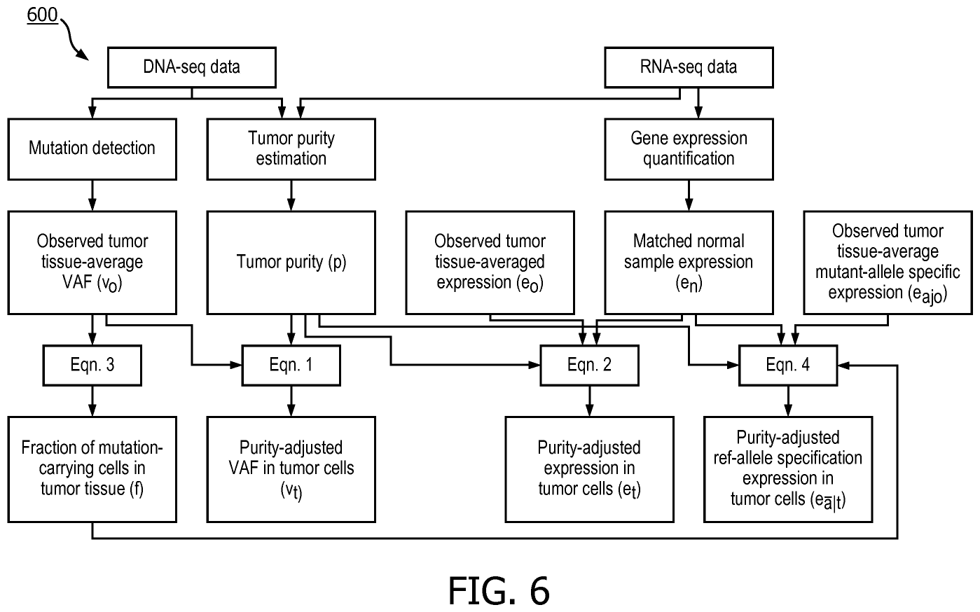

Referring to FIG. 6, in one embodiment, is a flowchart 600 for determination of tumor purity, according to one embodiment. A fast and effective method to suppress the noise signal introduced by the normal cells in genomic (e.g. mutational burden or VAF), transcriptomic (e.g. gene expression), epigenetic (e.g. methylation), proteomic or other quantitative data, enables a more accurate determination of their presence/abundance in the tumor cells and mitigates the confounding effect of tumor purity in subsequent data analysis.

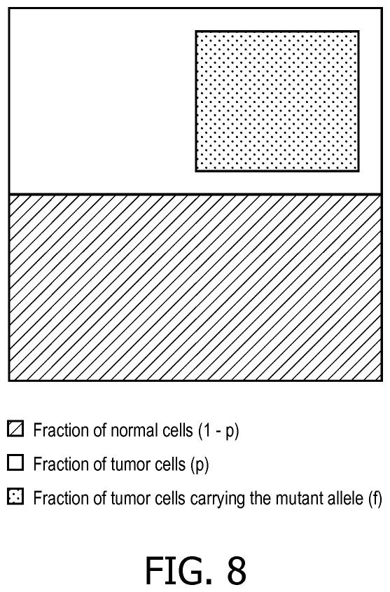

According to an embodiment, as schematically depicted in FIG. 8, a tumor sample/biopsy will comprise tumor cells with fraction p, i.e., tumor purity and normal cells (1−p). The fraction of tumor cells carrying a specific mutant allele is ƒ. According to an embodiment aimed at calculating tumor purity, certain variables can be determined and defined. It should be noted that tumor cells refer to the fraction of abnormal cells from the tumor tissue that consists of an admixture of tumor and normal cells.

According to an embodiment, for example, a tumor purity calculation can comprise the following variables for each somatic mutation: vₒ=the observed variant allele frequency of the mutant allele in the tumor tissue; and vₜ=the adjusted variant allele frequency of the mutant allele in the tumor cells. For each gene: eₙ=the normalized expression level in the normal cells, i.e. matched normal tissue; eₒ=the observed normalized expression level in the tumor tissue; e_{a|o}=the observed normalized allele specific expression level in the tumor tissue; eₜ=the adjusted normalized expression level in the tumor cells; e_{a|t}=the adjusted normalized mutant-allele specific expression level in the tumor cells; and e_{a|t}=the adjusted normalized reference-allele specific expression level in the tumor cells.

According to an embodiment, it can be assumed for purposes of the tumor purity calculation that: tumor purity p is known and can be estimated by a pathologist or computational analysis of genomic data; eₙ is the expression of normal cells obtained from matched normal tissue of the same patient or averaging over the normal tissues of a cohort of individuals; and vₒ, eₒ, and e_{a|o} are observed tissue-averaged data generated by bioinformatics tool from the DNA and RNA/proteomic data. VAF of a mutant allele in tumor cells can be calculated according to the following equation:

$$\begin{matrix}{v}_{t} = {v}_{o} / p & ( Eq . 4 ) \\ {e}_{o} = ( 1 - p ) {e}_{n} + p {e}_{t} ⇒ {e}_{t} = \frac{1}{p} \cdot [ {e}_{o} - ( 1 - p ) {e}_{n} ] & ( Eq . 5 )\end{matrix}$$

It can further be assumed for purposes of the tumor purity calculation that each cell only carries one copy of the mutation, then the fraction of cells in sample carrying the specific mutation is:

ƒ=2vₒ (Eq. 6)

It can further be assumed for purposes of the tumor purity calculation that a somatic mutant allele only exists in the tumor cells, then e_{a|t}=e_{a|o}. Since eₜ is also given by

$${e}_{t} = \frac{f}{p} . \\ {e}_{a \vert o} + \frac{p - f}{p} \cdot {e}_{\overline{a} \vert t} ,$$

based on Eq. (5), the following is arrived at:

$${e}_{t} = \frac{1}{p} \cdot [ {e}_{o} - ( 1 - p ) {e}_{n} ] = \frac{f}{p} \cdot {e}_{a \vert o} + \frac{p - f}{p} \cdot {e}_{\overline{a} \vert t} \\ ⇒ {e}_{\overline{a} \vert t} = \frac{1}{p - f} \cdot [ {e}_{o} - ( 1 - p ) {e}_{n} - f {e}_{a \vert o} ] \qquad ( Eq . 7 )$$

By applying Eqns. 4-7, one can adjust for tumor purity in the genomic and transcriptomic data and mitigate its confounding effect in subsequent data analysis.

Although this analysis primarily focuses on the adjustment for tumor purity, Eq. (5) can easily be generalized to support the adjustment for multiple cell subpopulations:

$${e}_{o} = {\sum }_{i = 1}^{k} ( {q}_{i} \cdot {e}_{i} ) ⇒ {e}_{t} = \frac{1}{{q}_{t}} [ {e}_{o} - {\sum }_{i \neq t} ( {q}_{i} \cdot {e}_{i} ) ] \qquad ( Eq . 8 )$$

where qᵢ and eᵢ are respectively the fraction of cells and gene expression in subpopulation i, k is the total number of subpopulations, t denotes the index of the target subpopulation whose expression profile needs to be estimated, and Σᵢ₌₁^{k}qᵢ=1.

According to an embodiment, the process may be utilized to adjust for or based on tumor purity. A first step may be to estimate the tumor purity p of the tissue sample. There are many existing computational tools and methods for this purpose based on the deconvolution of genomic and transcriptomic data. With matched normal sample available, somatic mutations can be identified by running variant callers, such as GATK, on the DNA sequencing data, and their observed variant allele frequencies (VAF) in the sample is simply calculated using the following formula:

$${v}_{o} = \frac{{t}_{-} a l {t}_{-} c o u n t}{{t}_{-} r e {f}_{-} c o u n t + {t}_{-} a l {t}_{-} c o u n t} \qquad ( Eq . 9 )$$

where t_ref_count is the number of reads carrying the reference allele and t_alt_count is the number of reads carrying the alternative/mutant allele in the tumor sample. Eq. 4 can then be applied to find their VAF in the tumor cells. These purity-adjusted VAF values are useful for the study and assessment of mutation burden and tumor progression.

By performing microarray or RNA sequencing, gene or protein expressions eₒ and eₙ can be obtained for respectively the tumor and matched normal tissues. If matched normal tissues are not available, one could also estimate eₙ by using the average expression of the normal tissues of other individuals. With known tumor purity, one can then compute the gene/protein expression in the tumor cells using Eq. 5. Such purity-adjusted expression data can improve the robustness of downstream analysis by removing the confounding effect of tumor purity.

For RNA sequencing data, one can further compute the allele specific expression (ASE) in the tumor tissue by using tools such as ASEReadCounter from GATK, AlleleSeq and Allim. One can then compute the reference-allele specific expression in the tumor cells by applying Eqs. 6 and 7. This can enable more effective investigate of the cis-acting effect of a mutation in the tumor cells by excluding any differential expression due to the difference between tumor and normal cells. The flowchart for the adjustment of tumor purity in genomic and transcriptomic data is shown in FIG. 6.

According to an embodiment, the process may be utilized to compute or otherwise analyze the gene expression of an emerging cell subpopulation. For example, two tissue biopsies can be obtained from the same site of a patient at two different time points, and there may be a need to investigate the gene expression profile of any new cell subpopulation that has emerged during the period. Assume a new somatic mutation is identified in the second sample with a VAF of vₒ and assume further that this mutation is tied only to the new subpopulation. In this case, the fraction of cells for the new subpopulation is estimated to be 2vₒ by Eq. 6, with the assumption that each cell only carries one copy of the mutant allele. The gene expression profile of the new subpopulation can then be obtained by applying Eqn. 5 as follows:

$$e = \frac{1}{2 {v}_{o}} \cdot [ {e}_{2} - ( 1 - 2 {v}_{o} ) {e}_{1} ] \qquad ( Eq . 9 )$$

where e₁ and e₂ are respectively the gene expression values at the first and second time points.

According to an embodiment, the process may be utilized to adjust for gene expression profiles of known cell types. For example, a target cell subpopulation t may be known to be contaminated by k other cell types, each with a well-defined gene expression signature. By means of deconvolution, one is able to estimate the fraction qᵢ of each cell type i. As an alternative, qᵢ may also be estimated by histology image analysis. Since the average expression profile eᵢ is known for each cell type i, we can compute the gene expression profile of the cell subpopulation of interest by applying Eq. 8 as follows:

$${e}_{t} = \frac{1}{{q}_{t}} [ {e}_{o} - {\sum }_{i \neq t} ( {q}_{i} \cdot {e}_{i} ) ] = \frac{1}{1 - {\sum }_{i \neq t} {q}_{i}} [ {e}_{o} - {\sum }_{i \neq t} ( {q}_{i} \cdot {e}_{i} ) ] \qquad ( Eq . 10 )$$

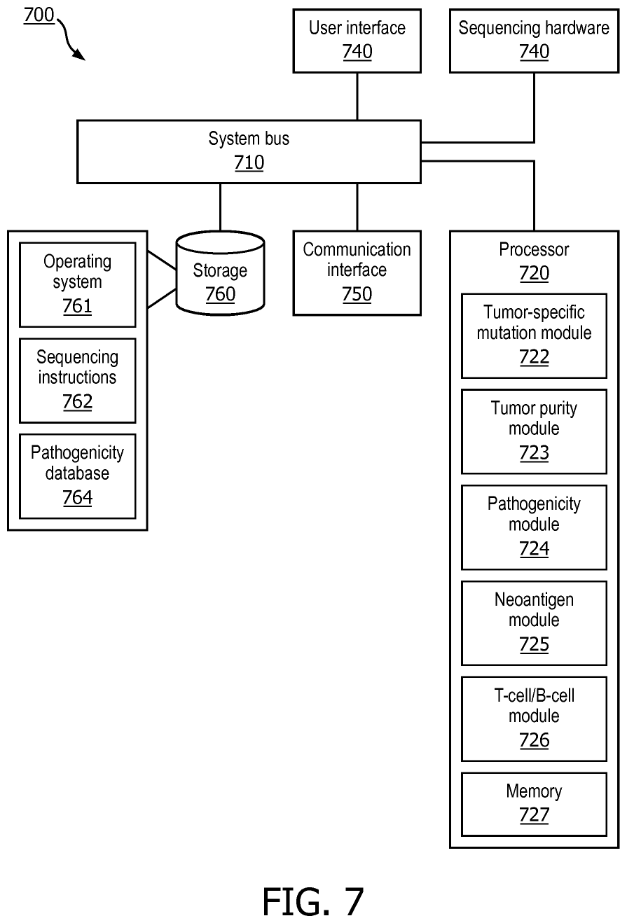

Referring to FIG. 7, in one embodiment, is a schematic representation of a system 700 for predicting the response of a tumor to immunotherapy. System 700 includes one or more of a processor 720, memory 727, user interface 740, communications interface 750, and storage 760, interconnected via one or more system buses 710. In some embodiments, such as those where the system comprises or implements a sequencer or sequencing platform, the hardware may include additional sequencing hardware 715, which may be any sequencer or sequencing platform. It will be understood that FIG. 7 constitutes, in some respects, an abstraction and that the actual organization of the components of the system 700 may be different and more complex than illustrated.

According to an embodiment, system 700 comprises a processor 720 capable of executing instructions stored in memory 727 or storage 760 or otherwise processing data. Processor 720 performs one or more steps of the method, and may comprise one or more of the modules described or otherwise envisioned herein. Processor 720 may be formed of one or multiple modules, and can comprise, for example, a memory 727. Processor 720 may take any suitable form, including but not limited to a microprocessor, microcontroller, multiple microcontrollers, circuitry, field programmable gate array (FPGA), application-specific integrated circuit (ASIC), a single processor, or plural processors.

Memory 727 can take any suitable form, including a non-volatile memory and/or RAM. The memory 727 may include various memories such as, for example a cache or system memory. As such, the memory 727 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 700. It will be apparent that, in embodiments where the processor implements one or more of the functions described herein in hardware, the software described as corresponding to such functionality in other embodiments may be omitted.

User interface 740 may include one or more devices for enabling communication with a user such as an administrator. The user interface can be any device or system that allows information to be conveyed and/or received, and may include a display, a mouse, and/or a keyboard for receiving user commands. In some embodiments, user interface 740 may include a command line interface or graphical user interface that may be presented to a remote terminal via communication interface 750. The user interface may be located with one or more other components of the system, or may located remote from the system and in communication via a wired and/or wireless communications network.

Communication interface 750 may include one or more devices for enabling communication with other hardware devices. For example, communication interface 750 may include a network interface card (NIC) configured to communicate according to the Ethernet protocol. Additionally, communication interface 750 may implement a TCP/IP stack for communication according to the TCP/IP protocols. Various alternative or additional hardware or configurations for communication interface 750 will be apparent.

Storage 760 may include one or more machine-readable storage media such as read-only memory (ROM), random-access memory (RAM), magnetic disk storage media, optical storage media, flash-memory devices, or similar storage media. In various embodiments, storage 760 may store instructions for execution by processor 720 or data upon which processor 720 may operate. For example, storage 760 may store an operating system 761 for controlling various operations of system 700. Where system 700 implements a sequencer and includes sequencing hardware 715, storage 760 may include sequencing instructions 762 for operating the sequencing hardware 715. According to an embodiment, storage 760 may include a pathogenicity database 764 as described or otherwise envisioned herein.

It will be apparent that various information described as stored in storage 760 may be additionally or alternatively stored in memory 727. In this respect, memory 727 may also be considered to constitute a storage device and storage 760 may be considered a memory. Various other arrangements will be apparent. Further, memory 727 and storage 760 may both be considered to be non-transitory machine-readable media. As used herein, the term non-transitory will be understood to exclude transitory signals but to include all forms of storage, including both volatile and non-volatile memories.

While system 700 is shown as including one of each described component, the various components may be duplicated in various embodiments. For example, processor 720 may include multiple microprocessors that are configured to independently execute the methods described herein or are configured to perform steps or subroutines of the methods described herein such that the multiple processors cooperate to achieve the functionality described herein. Further, where system 700 is implemented in a cloud computing system, the various hardware components may belong to separate physical systems. For example, processor 720 may include a first processor in a first server and a second processor in a second server. Many other variations and configurations are possible.

According to an embodiment, processor 720 comprises one or more modules to carry out one or more functions or steps of the methods described or otherwise envisioned herein. For example, processor 720 may comprise a tumor-specific mutation module 722 (identify & variant frequency), a tumor purity module 723, a pathogenicity module 724, a neoantigen module 725, and/or a T-cell/B-cell module 726, among other possible modules.

According to an embodiment, tumor-specific mutation module 722 identifies one or more tumor-specific mutations, and/or determines frequencies of tumor-specific mutations. Tumor-specific mutation module 722 can compare genetic information obtained from a tumor sample to genetic information obtained from a non-tumor sample to identify one or more mutations that are found only within the tumor sample. The tumor-specific mutation module 722 can also analyze the genetic information obtained from the tumor sample to determine a frequency of the identified mutations found only within the tumor sample. This information may be obtained during sequencing of the genetic material from the tumor sample, or may be obtained after sequencing by analyzing stored sequencing information. According to one embodiment, the allele frequency is determined or estimated by quantifying, tracking, or otherwise counting the percentage of reads that encompass the location of a mutation and that comprise the mutant allele, relative to the percentage of reads that encompass the location of a mutation and do not comprise the mutant allele. Many other methods for determining, estimating, or otherwise quantifying allele frequencies are possible.

According to an embodiment, processor 720 comprises a tumor purity module 723. Tumor purity module 723 analyzes the genetic information obtained from the tumor sample to determine or characterize a tumor purity of the patient's tumor. According to an embodiment, tumor purity can be estimated, calculated, or otherwise characterized by analysis of the genomic data by one or more algorithms. For example, an algorithm can be programmed, trained, or designed to calculate the most likely collection of genomes and their proportions in a sample using mutations, copy number aberrations, and/or other markers to distinguish between subpopulations.

According to an embodiment, processor 720 comprises a pathogenicity module 724. According to an embodiment, pathogenicity module 724 may calculate or retrieve a pathogenicity for the identified one or more tumor-specific mutations. For example, pathogenicity may be based on any available information about a mutation. Accordingly, pathogenicity module 724 may be in communication with a pathogenicity database such as pathogenicity database 764, which may be a component of system 700 or may be remote from system 700. Pathogenicity may also or alternatively be based on analysis of the mutation by pathogenicity module 724. For example, a mutation may not have pathogenicity information available, or may not have sufficient pathogenicity information available, but pathogenicity module 724 may determine that the mutation is sufficiently similar to another mutation such that the pathogenicity will also be similar.

According to an embodiment, processor 720 comprises a neoantigen module 725. According to an embodiment, neoantigen module 725 determines a neoantigen score for the identified tumor-specific mutations, where the neoantigen score comprises a likelihood that the mutation will be presented at the surface of tumor cells as a neoantigen. The neoantigen module 725 may utilize the patient's HLA type and/or bioinformatics tools such as EpiJen, WAPP, NetCTL, and/or NetCTLpan, among other tools or algorithms, to calculate the neoantigen score.

According to an embodiment, processor 720 comprises a T-cell/B-cell module 726. According to an embodiment, T-cell/B-cell module 726 determines a T-cell reactivity score for the identified tumor-specific mutations, where the T-cell reactivity score comprises a likelihood that the mutation will be recognized by the patient's T cells to induce an anti-tumor immune response. Among many other methods, the T-cell reactivity score can be calculated or inferred by the T-cell/B-cell module 726 using bioinformatics tools or algorithms such as POPI and/or POPISK, among others.

According to an embodiment, T-cell/B-cell module 726 determines a B-cell epitope score for the identified tumor-specific mutations, where the B-cell epitope score comprises a likelihood that the mutation will be recognized by the patient's B-cell receptors. Among many other methods, the B-cell epitope score can be calculated or inferred by the T-cell/B-cell module 726 using bioinformatics tools or algorithms such as COBEpro, BCPRed, and/or FBCPred for continuous-sequence epitopes (˜85% of documented B-cell epitopes), and EPMeta for discontinuous-sequence epitopes, among others.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for predicting a response of a tumor to immunotherapy, comprising the steps of:
analyzing a tumor sample obtained from a patient's tumor, comprising sequencing at least a portion of the genetic information of the tumor sample, wherein the tumor sample comprises a plurality of different genomes differentiated by one or more mutations, at least some of the mutations present at variable amounts within the tumor sample;
analyzing a non-tumor sample obtained from the patient, comprising sequencing at least a portion of the genetic information of the non-tumor sample;
identifying, by comparing the genetic information from the tumor sample to the genetic information from the non-tumor sample, one or more tumor-specific mutations found only in the tumor sample;
analyzing the genetic information from the tumor sample to determine a variant allele frequency for the identified one or more tumor-specific mutations;
analyzing the genetic information from the tumor sample to determine a tumor purity of the patient's tumor;
determining a pathogenicity for at least one of the identified one or more tumor-specific mutations;
calculating, from: (i) the determined variant allele frequency and/or a determined allele-specific expression, exon expression, or gene expression of the one or more tumor-specific mutations; (ii) the determined tumor purity; and (iii) the determined pathogenicity, a tumor functional mutation load score for the at least one of the identified one or more tumor-specific mutations;
predicting, based on the tumor functional mutation load score, a response of the patient's tumor to an immunotherapy treatment; and
determining, based on said prediction, a treatment for the patient.

2. The method of claim 1, wherein calculating the tumor functional mutation load score (Lₘ) comprises the equation:
$${L}_{m} = {\sum }_{i} [ f ( {v}_{i} , {a}_{i} , {e}_{i} ) \cdot {s}_{i} ]$$
where:
i is a tumor-specific mutation;
ƒ is a function measuring a presence or expression of a variant based on measurements vᵢ, aᵢ and eᵢ;
vᵢ is a determined variant allele frequency for the tumor-specific mutation i;
aᵢ is a determined allele-specific expression of mutation i for the tumor-specific mutation i;
eᵢ is a determined gene or exon expression of mutation i for the tumor-specific mutation i; and
sᵢ is the determined pathogenicity of the tumor-specific mutation i.

3. The method of claim 2, wherein one or more measurements of the equation are adjusted by a determined tumor purity of the tumor sample.

4. The method of claim 1, further comprising the step of obtaining a plurality of samples from the patient, including a sample from the patient's tumor and a non-tumor sample.

5. The method of claim 1, wherein the step of determining a pathogenicity for a tumor-specific mutation comprises querying a pathogenicity database.

6. A method for predicting a response of a tumor to immunotherapy, comprising the steps of:
analyzing a tumor sample obtained from a patient's tumor, comprising sequencing at least a portion of the genetic information of the tumor sample, wherein the tumor sample comprises a plurality of different genomes differentiated by one or more mutations, at least some of the mutations present at variable amounts within the tumor sample;
analyzing a non-tumor sample obtained from the patient, comprising sequencing at least a portion of the genetic information of the non-tumor sample;
identifying, by comparing the genetic information from the tumor sample to the genetic information from the non-tumor sample, one or more tumor-specific mutations found only in the tumor sample;
analyzing the genetic information from the tumor sample to determine a variant allele frequency for the identified one or more tumor-specific mutations
analyzing the genetic information from the tumor sample to determine a tumor purity of the patient's tumor;
determining one or more of: (i) a neoantigen score for the at least one of the identified one or more tumor-specific mutations, comprising a likelihood that the mutation will be presented as a neoantigen; (ii) a T-cell reactivity score for the at least one of the identified one or more tumor-specific mutations, comprising a likelihood that the mutation will be recognized by the patient's T cells; and (iii) a B-cell epitope score for the at least one of the identified one or more tumor-specific mutations, comprising a likelihood that the mutation will be recognized by the patient's B-cell receptors;
calculating, from: (i) the neoantigen score, the T-cell reactivity score, and/or the B-cell epitope score; (ii) the determined tumor purity; and (iii) the determined variant allele frequency and/or a determined allele-specific expression, exon expression, or gene expression of the identified one or more tumor-specific mutations, a tumor neoepitope load score for the at least one of the identified one or more tumor-specific mutations;
predicting, based on the tumor neoepitope load score, a response of the patient's tumor to an immunotherapy treatment; and
determining, based on said prediction, a treatment for the patient.

7. The method of claim 6, wherein calculating the tumor neoepitope load score (Lₙ) comprises the equation:
$${L}_{n} = {\sum }_{i} [ f ( {v}_{i} , {a}_{i} , {e}_{i} ) \cdot ( {n}_{i} \cdot {r}_{i} + {b}_{i} ) ]$$
where:
i is a tumor-specific mutation;
ƒ is a function measuring a presence or expression of a variant based on measurements vᵢ, aᵢ and eᵢ;
vᵢ is a determined variant allele frequency for the tumor-specific mutation i;
aᵢ is a determined allele-specific expression of mutation i for the tumor-specific mutation i;
eᵢ is a determined gene or exon expression of mutation i for the tumor-specific mutation i;
nᵢ is the neoantigen score;
rᵢ is the T-cell reactivity score; and
bᵢ is the B-cell epitope score.

8. The method of claim 7, wherein one or more measurements of the equation are adjusted by a determined tumor purity of the tumor sample.

9. The method of claim 6, further comprising the step of weighting a T-cell immune response for the tumor to produce a T-cell immune response weight, wherein the calculation of the tumor neoepitope load score further comprise the T-cell immune response weight.

10. The method of claim 9, further comprising the step of weighting a B-cell immune response for the tumor to produce a B-cell immune response weight, wherein the calculation of the tumor neoepitope load score further comprise the B-cell immune response weight.

11. The method of claim 10, wherein calculating the tumor neoepitope load score (Lₙ) comprises the equation:
$${L}_{n} = {\sum }_{i} [ f ( {v}_{i} , {a}_{i} , {e}_{i} ) \cdot ( {w}_{t} \cdot {n}_{i} \cdot {r}_{i} + {w}_{b} \cdot {b}_{i} ) ]$$
where:
i is a tumor-specific mutation;
ƒ is a function measuring a presence or expression of a variant based on measurements vᵢ, aᵢ and eᵢ;
vᵢ is a determined variant allele frequency for the tumor-specific mutation i;
aᵢ is a determined allele-specific expression of mutation i for the tumor-specific mutation i;
eᵢ is a determined gene or exon expression of mutation i for the tumor-specific mutation i;
nᵢ is the neoantigen score;
rᵢ is the T-cell reactivity score;
bᵢ is the B-cell epitope score;
wₜ is the T-cell immune response weight; and
w_{b} is the B-cell immune response weight.

12. The method of claim 11, wherein one or more measurements of the equation are adjusted by a determined tumor purity of the tumor sample.

13. A system configured to predict a response of a tumor to immunotherapy, comprising:
a processor configured to: (i) identify, by comparing genetic information from a tumor sample to genetic information from a non-tumor sample, one or more tumor-specific mutations found only in the tumor sample; (ii) analyze the genetic information from the tumor sample to determine a variant allele frequency for the identified one or more tumor-specific mutations; (iii) analyze the genetic information from the tumor sample to determine a tumor purity of the patient's tumor; (iv) determine a pathogenicity for at least one of the identified one or more tumor-specific mutations; (v) calculate, from: (i) the determined variant allele frequency and/or a determined allele-specific expression, exon expression, or gene expression of the one or more tumor-specific mutations; (ii) the determined tumor purity; and (iii) the determined pathogenicity, a tumor functional mutation load score for the at least one of the identified one or more tumor-specific mutations; and (vi) predict, based on the tumor functional mutation load score, a response of the patient's tumor to an immunotherapy treatment; and
a user interface configured to provide said prediction to a user.

14. The system of claim 13, wherein the processor is configured to calculate the tumor functional mutation load score (Lₙ) using the equation:
$${L}_{m} = {\sum }_{i} [ f ( {v}_{i} , {a}_{i} , {e}_{i} ) \cdot {s}_{i} ]$$
where:
i is a tumor-specific mutation;
ƒ is a function measuring a presence or expression of a variant based on measurements vᵢ, aᵢ and eᵢ,
vᵢ is a determined variant allele frequency for the tumor-specific mutation i;
aᵢ is a determined allele-specific expression of mutation i for the tumor-specific mutation i;
eᵢ is a determined gene or exon expression of mutation i for the tumor-specific mutation i; and
sᵢ is the determined pathogenicity of the tumor-specific mutation i.

15. The system of claim 14, wherein one or more measurements of the equation are adjusted by a determined tumor purity of the tumor sample.

16. The system of claim 13, further comprising a pathogenicity database, and wherein the processor is configured to determine a pathogenicity using data from the pathogenicity database.

17. A system configured to predict a response of a tumor to immunotherapy, comprising:
a processor configured to: (i) identify, by comparing genetic information from a tumor sample to genetic information from a non-tumor sample, one or more tumor-specific mutations found only in the tumor sample; (ii) analyze the genetic information from the tumor sample to determine a variant allele frequency for the identified one or more tumor-specific mutations; (iii) analyze the genetic information from the tumor sample to determine a tumor purity of the patient's tumor; (iv) determine a neoantigen score for the at least one of the identified one or more tumor-specific mutations, comprising a likelihood that the mutation will be presented as a neoantigen; (v) determine a T-cell reactivity score for the at least one of the identified one or more tumor-specific mutations, comprising a likelihood that the mutation will be recognized by the patient's T cells; (vi) determine B-cell epitope score for the at least one of the identified one or more tumor-specific mutations, comprising a likelihood that the mutation will be recognized by the patient's B-cell receptors; (vii) calculate, from: (i) the neoantigen score, the T-cell reactivity score, and/or the B-cell epitope score; (ii) the determined tumor purity; and (iii) the determined variant allele frequency and/or a determined allele-specific expression, exon expression, or gene expression of the identified one or more tumor-specific mutations, a tumor neoepitope load score for the at least one of the identified one or more tumor-specific mutations; and (viii) predict, based on the tumor neoepitope load score, a response of the patient's tumor to an immunotherapy treatment; and
a user interface configured to provide said prediction to a user.

18. The system of claim 17, wherein the processor is configured to calculate the tumor neoepitope load score (Lₙ) using the equation:
$${L}_{n} = {\sum }_{i} [ f ( {v}_{i} , {a}_{i} , {e}_{i} ) \cdot ( {n}_{i} \cdot {r}_{i} + {b}_{i} ) ]$$
where:
i is a tumor-specific mutation;
ƒ is a function measuring a presence or expression of a variant based on measurements vᵢ, aᵢ and eᵢ;
vᵢ is a determined variant allele frequency for the tumor-specific mutation i;
aᵢ is a determined allele-specific expression of mutation i for the tumor-specific mutation i;
eᵢ is a determined gene or exon expression of mutation i for the tumor-specific mutation i;
nᵢ is the neoantigen score;
rᵢ is the T-cell reactivity score; and
bᵢ is the B-cell epitope score.

19. The system of claim 18, wherein one or more measurements of the equation are adjusted by a determined tumor purity of the tumor sample.
